(19) United States
(12) Patent Application Publication
(10) Pub. No.: US 2025/0352377 A1
(43) Pub. Date: Nov. 20, 2025

(54) INTRODUCER SHEATH WITH DEPLOYABLE GRAFT

(71) Applicant: BOSTON SCIENTIFIC SCIMED, INC., MAPLE GROVE, MN (US)

(72) Inventors: Daniel Presteng, Corcoran, MN (US); Benjamin Jerome Bidne, Loretto, MN (US); Jeffrey Schneider, Maple Grove, MN (US); Zachary Kumor, Plymouth, MI (US); Lauren Ann Sandy, Minneapolis, MN (US)

(73) Assignee: BOSTON SCIENTIFIC SCIMED, INC., MAPLE GROVE, MN (US)

(21) Appl. No.: 19/212,423

(22) Filed: May 19, 2025

Related U.S. Application Data
(60) Provisional application No. 63/649,531, filed on May 20, 2024.

(51) Int. Cl. A61F 2/966

(52) U.S. Cl. CPC A61F 2/966; A61F 2002/9665

(57) ABSTRACT

An access system for accessing a patient's vasculature includes an introducer sheath that is adapted to extend into an interior of a blood vessel through an opening formed in a wall of the blood vessel in order to provide access to the interior of the blood vessel. A deployable graft is disposed relative to the distal region of the introducer sheath. One or more positioning elements are releasably secured to the deployable graft and extend proximally therefrom such that exerting a force on the one or more positioning elements causes the deployable graft to move proximally within the interior of the blood vessel to a position in which the deployable graft spans the opening formed in the wall of the blood vessel. An outer jacket releasably secures the deployable graft in position relative to the introducer sheath.

### CROSS-REFERENCE TO RELATED APPLICATION

This application is a continuation of U.S. Patent Application Ser. No. 63/649,531, filed May 20, 2024, entitled “INTRODUCER SHEATH WITH DEPLOYABLE GRAFT”, which is incorporated by reference herein in its entirety.

### TECHNICAL FIELD

The disclosure relates generally to medical devices and more particularly to introducer sheaths.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in an access system for accessing a patient's vasculature. The access system includes an introducer sheath that is adapted to extend into an interior of a blood vessel through an opening formed in a wall of the blood vessel in order to provide access to the interior of the blood vessel, the introducer sheath including a distal region. A deployable graft is disposed relative to the distal region of the introducer sheath. One or more positioning elements are releasably secured to the deployable graft and extend proximally therefrom such that exerting a force on the one or more positioning elements causes the deployable graft to move proximally within the interior of the blood vessel to a position in which the deployable graft spans the opening formed in the wall of the blood vessel.

Alternatively or additionally, the one or more positioning elements may be releasably secured to the deployable graft at a position distal of a proximal end of the deployable graft.

Alternatively or additionally, the deployable graft may include a distal portion, a proximal portion and an intervening middle portion, and wherein the one or more positioning elements may be secured to the middle portion of the deployable graft.

Alternatively or additionally, the one or more positioning elements may include a suture having two ends that each extend proximally to a point at which the two ends of the suture can be manipulated.

Alternatively or additionally, the access system may further include an outer jacket adapted to extend over the distal region of the introducer sheath and the deployable graft in order to hold the deployable graft in position relative to the distal region of the introducer sheath.

Alternatively or additionally, withdrawing the outer jacket proximally may free the deployable graft to move relative to distal region of the introducer sheath.

Alternatively or additionally, the deployable graft may include a stent.

Alternatively or additionally, the deployable graft may include a framework and an occlusive cover covering the framework.

Another example may be found in an access system for accessing a patient's vasculature. The access system includes an introducer sheath including a distal region, a deployable graft releasably secured relative to the distal region of the introducer sheath, and an outer jacket adapted to extend over the distal region of the introducer sheath and the deployable graft in order to releasably secure the deployable graft relative to the distal region of the introducer sheath. Withdrawing the outer jacket proximally frees the deployable graft to move relative to the distal region of the introducer sheath.

Alternatively or additionally, the access system may further include one or more positioning elements releasably secured to the deployable graft and extending proximally therefrom such that exerting a force on the one or more positioning elements can position the deployable graft within the interior of the blood vessel.

Alternatively or additionally, the one or more positioning elements may be releasably secured to the deployable graft at a position distal of a proximal end of the deployable graft.

Alternatively or additionally, the deployable graft may include a distal portion, a proximal portion and an intervening middle portion, and wherein the one or more positioning elements may be secured to the middle portion of the deployable graft.

Alternatively or additionally, the one or more positioning elements may extend proximally to a point at which the one or more positioning elements can be manipulated.

Alternatively or additionally, the deployable graft may include a stent.

Alternatively or additionally, the deployable graft may include a framework and an occlusive cover covering the framework.

Another example may be found in an access system for accessing a patient's vasculature. The access system includes an introducer sheath including a distal region, a deployable graft secured relative to the distal region of the introducer sheath, an outer jacket adapted to hold the deployable graft in position relative to the distal region of the introducer sheath, and one or more positioning elements that are releasably secured to the deployable graft and that extend proximally therefrom such that exerting a force on the one or more positioning elements translates the deployable graft within the interior of the blood vessel.

Alternatively or additionally, the outer jacket may have an initial position in which the outer jacket overlies at least part of the deployable graft, thereby holding the deployable graft relative to the introducer sheath.

Alternatively or additionally, withdrawing the outer jacket from its initial position may free the deployable graft and allows translation of the deployable graft.

Alternatively or additionally, the deployable graft may extend proximally from where the one or more positioning elements are releasably secured to the deployable graft.

Alternatively or additionally, the deployable graft may include a framework and an occlusive cover covering the framework.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic view of an illustrative access system for accessing a patient's vasculature;

FIG. 2 is a cross-sectional view taken along the line 2-2 of FIG. 1;

FIG. 3 is another schematic view of the illustrative access system of FIG. 1;

FIG. 4 is a schematic view of the illustrative access system of FIG. 3, with an outer jacket retracted;

FIG. 5 is a schematic view of the illustrative access system of FIG. 1 shown positioned within the vasculature, with the outer jacket partially withdrawn;

FIG. 6 is a schematic view of the illustrative access system of FIG. 1 shown positioned with the vasculature, with the outer jacket completely withdrawn and the positioning elements manipulated to move the deployable graft;

FIGS. 7, 8 and 9 together schematically show using the positioning elements to position the deployable graft;

FIG. 10 is a schematic view of the deployable graft; and

FIG. 11 is a cross-sectional view taken along the line 11-11 of FIG. 10.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Introducer sheathes may be used to provide access to an interior of a patient's vasculature in order to allow various medical devices such as but not limited to guidewires and various catheters to be advanced through the introducer sheath, used for their intended purpose, and then be withdrawn. After the procedure, the introducer sheath may also be withdrawn. This can leave a hole formed in the wall of the vessel where the introducer sheath penetrated the vessel wall. This hole needs to be closed in order to prevent bleeding. An access system for accessing a patient's vasculature may include an introducer sheath that is adapted to extend into an interior of a blood vessel through an opening formed in a wall of the blood vessel in order to provide access to the interior of the blood vessel. The access system also includes a deployable graft that is disposed relative to a distal region of the introducer sheath and one or more positioning elements that are releasably secured to the deployable graft and extend proximally therefrom such that exerting a force on the one or more positioning elements causes the deployable graft to move proximally within the interior of the blood vessel to a position in which the deployable graft spans the opening formed in the wall of the blood vessel.

In some cases, the one or more positioning elements may be releasably secured to the deployable graft at a position distal of a proximal end of the deployable graft. In some cases, the deployable graft may include a distal portion, a proximal portion and an intervening middle portion, and the one or more positioning elements may be secured to the middle portion of the deployable graft. In some cases, the one or more positioning elements may extend proximally to a point at which the one or more positioning elements can be manipulated.

In some cases, the access system may further include an outer jacket that is adapted to extend over the distal region of the introducer sheath and the deployable graft in order to hold the deployable graft in position relative to the distal region of the introducer sheath. Withdrawing the outer jacket proximally may free the deployable graft to move relative to distal region of the introducer sheath. The deployable graft may include a stent. The deployable graft may include a framework and an occlusive cover that covers the framework.

An access system for accessing a patient's vasculature includes an introducer sheath and a deployable graft that is releasably secured relative to the distal region of the introducer sheath. An outer jacket is adapted to extend over the distal region of the introducer sheath and the deployable graft in order to releasably secure the deployable graft relative to the distal region of the introducer sheath. Withdrawing the outer jacket proximally frees the deployable graft to move relative to the distal region of the introducer sheath.

In some cases, the access system may further include one or more positioning elements releasably secured to the deployable graft and extending proximally therefrom such that exerting a force on the one or more positioning elements can position the deployable graft within the interior of the blood vessel. In some cases, the one or more positioning elements may be releasably secured to the deployable graft at a position distal of a proximal end of the deployable graft. In some cases, the deployable graft includes a distal portion, a proximal portion and an intervening middle portion, and the one or more positioning elements may be secured to the middle portion of the deployable graft. The one or more positioning elements may extend proximally to a point at which the one or more positioning elements can be manipulated. The deployable graft may include a stent. The deployable graft may include a framework and an occlusive cover that covers the framework.

An access system for accessing a patient's vasculature includes an introducer sheath, a deployable graft that is secured relative to a distal region of the introducer sheath, an outer jacket that is adapted to hold the deployable graft in position relative to the distal region of the introducer sheath, and one or more positioning elements that are releasably secured to the deployable graft and extend proximally therefrom such that exerting a force on the one or more positioning elements translates the deployable graft within the interior of the blood vessel. In some cases, the outer jacket may have an initial position in which the outer jacket overlies at least part of the deployable graft, thereby holding the deployable graft relative to the introducer sheath. Withdrawing the outer jacket from its initial position may free the deployable graft and may allow translation of the deployable graft. In some cases, the deployable graft may extend proximally from where the one or more positioning elements are releasably secured to the deployable graft. In some cases, the deployable graft may include a framework and an occlusive cover spanning the framework.

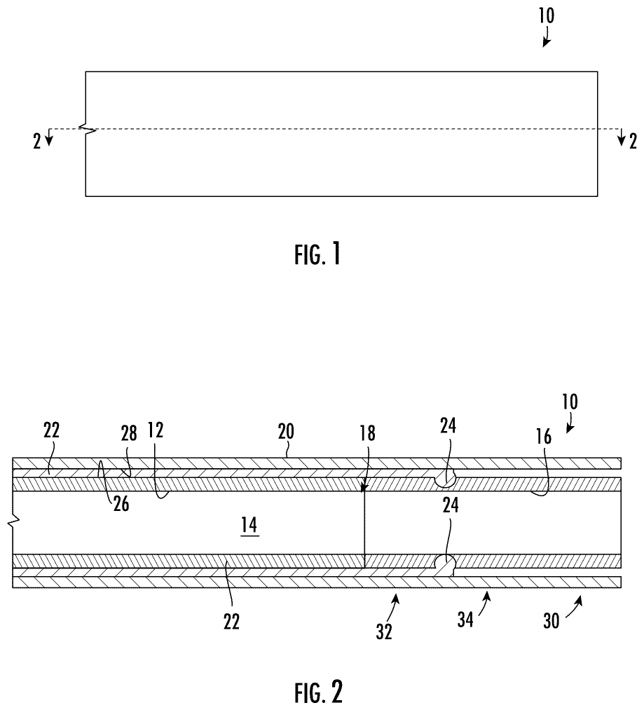

FIG. 1 is a schematic view of an illustrative access system 10 that may be used for accessing a patient's vasculature. FIG. 2 is a cross-sectional view of the illustrative access system 10, taken along the line 2-2 of FIG. 1. The access system 10 may be adapted to allow a physician or other professional to advance one, two or more different medical devices such as but not limited to guidewires and various catheters through the access system 10 and thus into the patient's vasculature so that the medical devices may reach a desired treatment site within the vasculature, and perform any of a variety of different medical treatments. The various medical devices may then be withdrawn after they have performed their assigned tasks in whatever medical protocol the physician or other professional desires to perform. The access system 10 may be used to provide access to any particular part of the patient's vasculature. In some cases, the access system 10 may be used to provide access to the patient's femoral artery, although other arteries may also be utilized.

The access system 10 includes an introducer sheath 12 that defines a lumen 14 extending through the introducer sheath 12. The lumen 14 may be dimensioned to allow a variety of different medical devices to be extended through the introducer sheath 12 and then subsequently withdrawn from the introducer sheath 12. In some cases, the lumen 14 may allow a single medical device to extend through the lumen 14 at one time. In some cases, the lumen 14 may allow two or more different medical devices to extend through the lumen 14 at one time. In some cases, the two or more different medical devices may include coaxial devices, such as a catheter advanced over a guidewire. The access system 10 includes a deployable graft 16 that is disposed adjacent a distal region 18 of the introducer sheath 12. In some cases, the deployable graft 16 may be releasably secured adjacent the distal region 18 of the introducer sheath 12 via an outer jacket 20 that extends over the introducer sheath 12 and the deployable graft 16. The outer jacket 20 may be formed of any suitable polymeric material. While the deployable graft 16 is shown as being positioned end-to-end with the introducer sheath 12, in some cases the deployable graft 16 may be positioned such that at least part of the deployable graft 16 overlies the distal region 18 of the introducer sheath 12.

In some cases, the access system 10 may include one or more (two are shown) positioning elements 22 that extend proximally from connection points 24 disposed on the deployable graft 16. The access system 10 may include a single positioning element 22. The access system 10 may include three, four or more positioning elements 22. In some cases, the positioning elements 22 extend proximally a distance that allows the positioning elements 22 to be manipulated from a position outside of the patient. In some cases, the positioning elements 22 may extend to a proximal hub (not shown). In some cases, as shown, the positioning elements 22 may extend proximally within a void space between an outer surface 26 of the introducer sheath 12 and an inner surface of the outer jacket 20. In some cases, the positioning elements 22 may extend within the lumen 14. In some case, the positioning elements 22 may extend exterior to the outer jacket 20, particularly if the outer jacket 20 does not extend all the way to a distal end of the deployable graft 16. The positioning elements 22 may be formed of any suitable material. In some cases, the positioning elements 22 may be formed of a metal such as stainless steel, for example.

As noted, the positioning elements 22 extend proximally from the connection points 24. In some cases, the connection points 24 may be positioned distally of a proximal end of the deployable graft 16. In some cases, the deployable graft 16 may be considered as including a distal portion 30, a proximal portion 32 and an intervening middle portion 34. In some cases, the connection points 24 may be positioned within the middle portion 34. By positioning the connection points 24 within the middle portion 34, or at least distal of the proximal end of the deployable graft 16, it is possible to pull the deployable graft 16 proximally such that the deployable graft 16 spans an opening formed in the blood vessel wall even though the positioning elements 22 are extending through that opening. In some cases, the positioning elements 22 may extend proximally to a point at which the positioning elements 22 may be manipulated.

In some cases, the connection points 24 may be secured to the deployable graft 16, and the positioning elements 22 may include a release mechanism (not shown). In some instances, the connection points 24 may themselves be formed of a radiopaque metal that allows the connection points 24 to serve as radiopaque markers. This can help in properly positioning the deployable graft 16 within the blood vessel in order to occlude the opening formed in the vessel wall to initially accommodate the access system 10. In some cases, the connection points 24 may schematically represent spots where the positioning elements 22 are secured to the deployable graft 16. In some cases, the connection points 24 may schematically represent where the positioning elements 22 are tied to the deployable graft 16. In some cases, the connection points 24 may schematically represent where the positioning elements 22 are hooked to the deployable graft 16. As an example, the distal end of each positioning element 22 may be hook-shaped, such that pulling a particular positioning element proximally allows the particular positioning elements 22 to exert a tensile force on the deployable graft 16, but pushing the particular positioning elements 22 allows that positioning element 22 to moved out of coupling with the deployable graft 16.

In some cases, the positioning elements 22 may represent halves of a suture and the connection points 24 may schematically represent where the suture passes through or otherwise engage a portion of the deployable graft 16. With the two halves of the suture extending proximally and out of the body, a physician or other professional may urge the deployable graft 16 proximally within the blood vessel by pulling on both halves of the suture. Once the deployable graft 16 has been appropriately positioned, the suture may be withdrawn by letting go of one end of the suture and pulling on the other end of the suture to pull the suture free of the deployable graft 16. These are just examples.

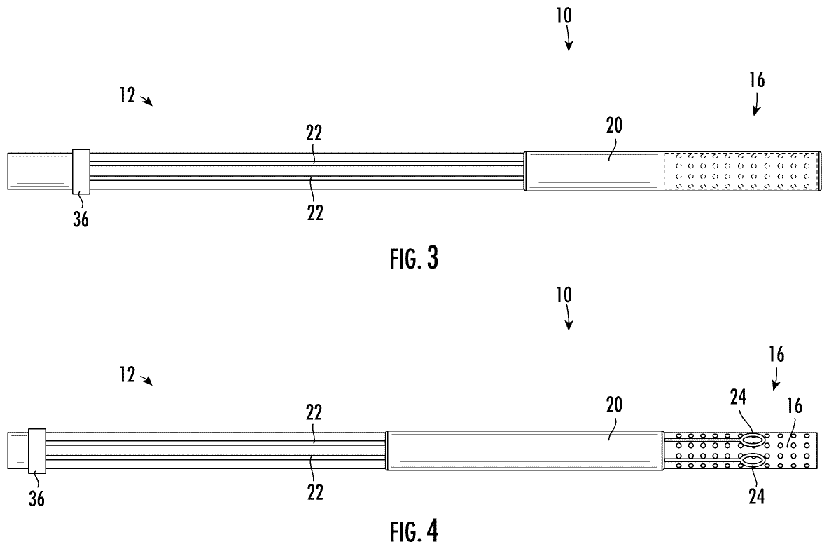

FIG. 3 is a schematic view of the access system 10. As shown, the positioning elements 22 extend proximally to a positioning element ring 36 that is disposed about the introducer sheath 12. In some cases, the positioning element ring 36 provides a way for the positioning elements 22 to be moved in unison, in order to keep the deployable graft 16 coaxial or at least substantially coaxial (defined as within ten percent of coaxial) with the introducer sheath 12. This can enable the deployable graft 16 able to move within the blood vessel in response to forces applied by the positioning elements 22 without getting stuck within the blood vessel as a result of the deployable graft 16 coming out of coaxial alignment with the introducer sheath 12.

As seen in FIG. 3, the outer jacket 20 extends distally over the deployable graft 16, which is shown in phantom underneath part of the outer jacket 20. FIG. 3 shows the access system 10 as it would be used initially as an introducer sheath that extends through the patient's skin, through an opening in a blood vessel, and into an interior of the blood vessel such that other medical devices may be advanced through the access system 10. The other medical devices may then be withdrawn from the access system 10 after performing the steps of a particular medical protocol, and the access system 10 may be prepared to deploy the deployable graft 16, as shown in FIG. 4. FIG. 4 shows the access system 10 with the outer jacket 20 partially withdrawn proximally. As a result, at least part of the deployable graft 16 has been exposed. The connection points 24 are visible on the deployable graft 16, where the positioning elements 22 are secured to the deployable graft.

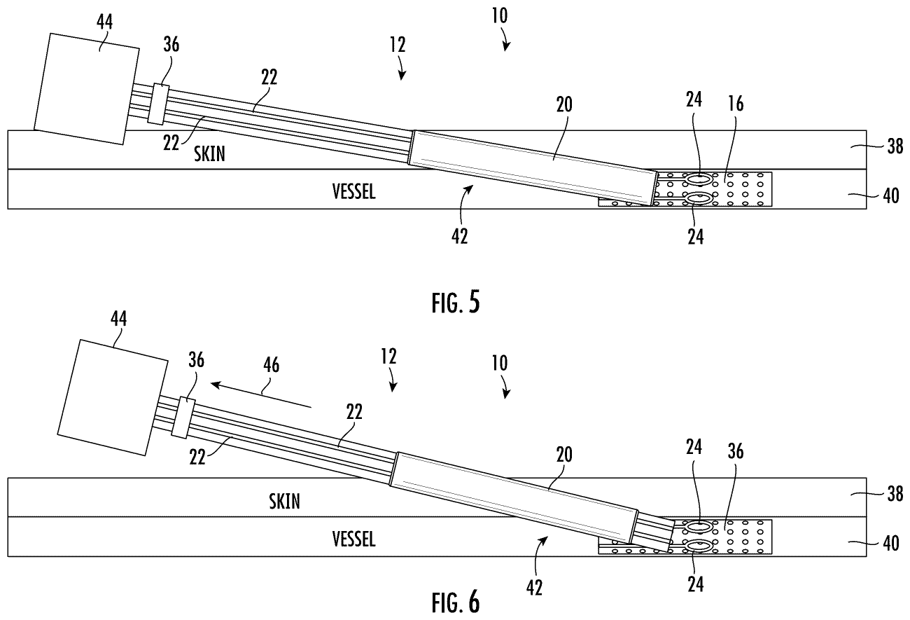

FIG. 5 shows the access system 10 extending through a skin layer 38 and into a blood vessel 40. It will be appreciated that an opening 42 has been formed within a vessel wall of the blood vessel 40 in order to allow the access system 10 to extend through the skin layer 38 and into an interior of the blood vessel 40. In FIG. 5, the outer jacket 20 has been partially withdrawn proximally in order to expose the deployable graft 16. As seen, the positioning elements 22 are secured to the connection points 24 so that the deployable graft 16 may be properly positioned relative to the opening 42 within the vessel wall of the blood vessel 40. The access system 10 includes a hub 44. In some cases, the hub 44 may include a mechanism that can be used to withdraw the outer jacket 20. In some cases, the hub 44 may include a mechanism that can be used to withdraw the positioning elements 22. In some instances, the positioning element ring 36 may instead be manipulated distal of the hub 44 in order to cause the deployable graft 16 to translate relative to the introducer sheath 12.

In FIG. 6, the outer jacket 20 has been fully retracted. The positioning elements 22 may now be used to properly position the deployable graft 16 relative to the opening 42 such that the deployable graft 16 spans the opening 42. The deployable graft 16 may be moved proximally by moving the positioning elements 22 and/or the positioning element ring 36 in a direction indicated by an arrow 46. Once the deployable graft 16 has been positioned to span the opening 42, the deployable graft 16 may be considered as being positioned to occlude the opening 42. This can reduce or even eliminate the need for other vascular closure devices such as vascular plugs or suturing techniques.

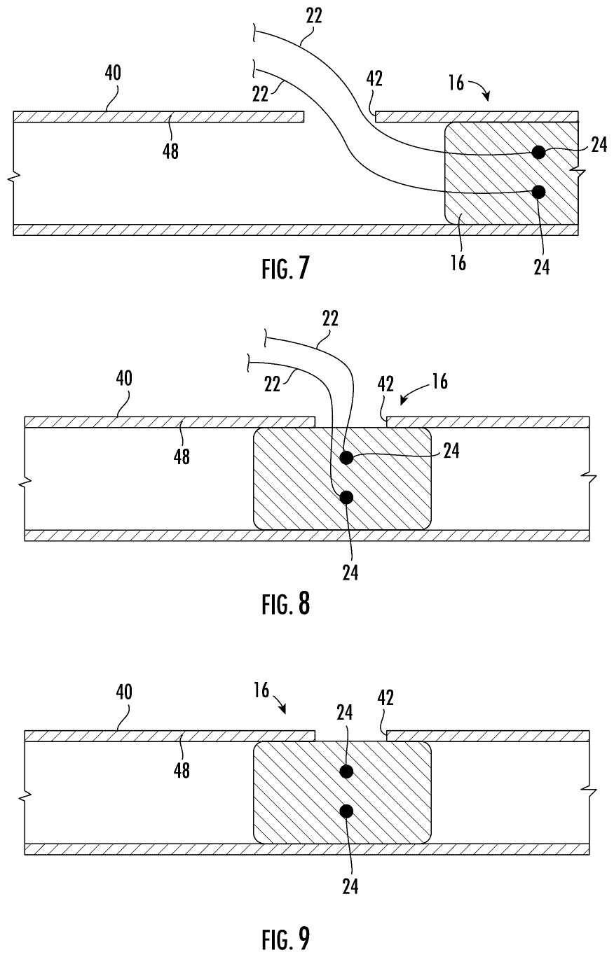

Positioning the deployable graft 16 may be explained with reference to FIGS. 7, 8 and 9. In these Figures, the rest of the access system 10 has been excluded for clarity. The blood vessel 40 has a vessel wall 48, and the opening 42 extends through the vessel wall 48. In FIG. 7, the deployable graft 16 is positioned distal of the opening 42, and may represent a position for the deployable graft 16 that corresponds to before the outer jacket 20 (FIGS. 2-6) has been withdrawn proximally to expose the deployable graft 16. Moving from FIG. 7 to FIG. 8, it can be seen that the deployable graft 16 has moved proximally and now spans the opening 42. As a result, the deployable graft 16 also occludes the opening 42. In some cases, the deployable graft 16 may increase slightly in diameter when no longer constrained by the outer jacket 20 extending over the deployable graft 16. In some cases, the deployable graft 16 moves proximally (such as from its position in FIG. 7 to its position in FIG. 8) as a result of a tensile force being applied by the positioning elements 22. Finally, and as shown in FIG. 9, the positioning elements 22 may be disconnected from the connection points 24, and the rest of the access system 10, apart from the deployable graft 16, may be withdrawn from the patient. The deployable graft 16 remains within the blood vessel 40, spanning and thus occluding the opening 42.

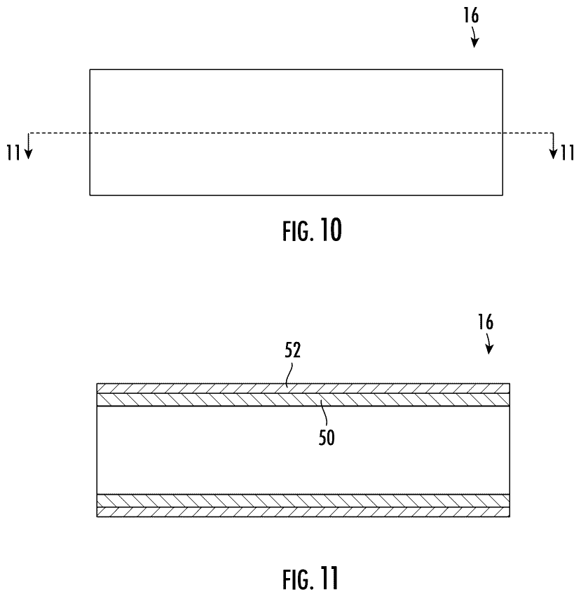

FIG. 10 is a schematic view of the deployable graft 16 and FIG. 11 is a cross-sectional view of the deployable graft 16 taken along the line 11-11 of FIG. 10. In some cases, the deployable graft 16 may be, or may include, a stent. The deployable graft 16 may be, or may include, a braided stent, a woven stent, a laser cut stent or even a polymeric stent. In some cases, as shown, the deployable graft 16 may include a framework 50 and an occlusive cover 52 that covers at least part of the framework 50. The framework 50 may include a braided framework, a woven framework or even a laser cut framework. The occlusive cover 52 may be a fabric or a polymeric covering that is impermeable to blood such that when the deployable graft 16 is disposed within the blood vessel 40 spanning the opening 42, the occlusive cover 52 prevents blood from flowing out of the opening 42.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, the devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An access system for accessing a patient's vasculature, the access system comprising:
an introducer sheath adapted to extend into an interior of a blood vessel through an opening formed in a wall of the blood vessel in order to provide access to the interior of the blood vessel, the introducer sheath including a distal region;
a deployable graft disposed relative to the distal region of the introducer sheath; and
one or more positioning elements releasably secured to the deployable graft and extending proximally therefrom such that exerting a force on the one or more positioning elements causes the deployable graft to move proximally within the interior of the blood vessel to a position in which the deployable graft spans the opening formed in the wall of the blood vessel.

2. The access system of claim 1, wherein the one or more positioning elements are releasably secured to the deployable graft at a position distal of a proximal end of the deployable graft.

3. The access system of claim 1, wherein the deployable graft includes a distal portion, a proximal portion and an intervening middle portion, and wherein the one or more positioning elements are secured to the middle portion of the deployable graft.

4. The access system of claim 1, wherein the one or more positioning elements comprise a suture having two ends that each extend proximally to a point at which the two ends of the suture can be manipulated.

5. The access system of claim 1, further comprising an outer jacket adapted to extend over the distal region of the introducer sheath and the deployable graft in order to hold the deployable graft in position relative to the distal region of the introducer sheath.

6. The access system of claim 5, wherein withdrawing the outer jacket proximally frees the deployable graft to move relative to distal region of the introducer sheath.

7. The access system of claim 1, wherein the deployable graft comprises a stent.

8. The access system of claim 1, wherein the deployable graft includes a framework and an occlusive cover covering the framework.

9. An access system for accessing a patient's vasculature, the access system comprising:
an introducer sheath including a distal region;
a deployable graft releasably secured relative to the distal region of the introducer sheath; and
an outer jacket adapted to extend over the distal region of the introducer sheath and the deployable graft in order to releasably secure the deployable graft relative to the distal region of the introducer sheath;
wherein withdrawing the outer jacket proximally frees the deployable graft to move relative to the distal region of the introducer sheath.

10. The access system of claim 9, further comprising one or more positioning elements releasably secured to the deployable graft and extending proximally therefrom such that exerting a force on the one or more positioning elements can position the deployable graft within the interior of the blood vessel.

11. The access system of claim 9, wherein the one or more positioning elements are releasably secured to the deployable graft at a position distal of a proximal end of the deployable graft.

12. The access system of claim 9, wherein the deployable graft includes a distal portion, a proximal portion and an intervening middle portion, and wherein the one or more positioning elements are secured to the middle portion of the deployable graft.

13. The access system of claim 9, wherein the one or more positioning elements extend proximally to a point at which the one or more positioning elements can be manipulated.

14. The access system of claim 9, wherein the deployable graft comprises a stent.

15. The access system of claim 9, wherein the deployable graft includes a framework and an occlusive cover covering the framework.

16. An access system for accessing a patient's vasculature, the access system comprising:
an introducer sheath including a distal region;
a deployable graft secured relative to the distal region of the introducer sheath;
an outer jacket adapted to hold the deployable graft in position relative to the distal region of the introducer sheath; and
one or more positioning elements releasably secured to the deployable graft and extending proximally therefrom such that exerting a force on the one or more positioning elements translates the deployable graft within the interior of the blood vessel.

17. The access system of claim 16, wherein the outer jacket has an initial position in which the outer jacket overlies at least part of the deployable graft, thereby holding the deployable graft relative to the introducer sheath.

18. The access system of claim 17, wherein withdrawing the outer jacket from its initial position frees the deployable graft and allows translation of the deployable graft.

19. The access system of claim 16, wherein the deployable graft extends proximally from where the one or more positioning elements are releasably secured to the deployable graft.

20. The access system of claim 16, wherein the deployable graft includes a framework and an occlusive cover covering the framework.
